(19) United States
(12) Patent Application Publication
(10) Pub. No.: US 2018/0338810 A1
(43) Pub. Date: Nov. 29, 2018

(54) IV POLE ISOLATION AND CLEANING DEVICES AND METHODS

(71) Applicant: Merit Medical Systems, Inc., South Jordan, UT (US)

(72) Inventors: Fred Lampropoulos, Salt Lake City, UT (US); Jim Mottola, West Jordan, UT (US); Kenneth Sykes, Bluffdale, UT (US); Christopher Cindrich, Highland, UT (US); Mahender Avula, Salt Lake City, UT (US); Bruce Lewis, Park City, UT (US); Richard P. Jenkins, Bluffdale, UT (US); John Hellgeth, Reno, NV (US); Brian Harris, Salt Lake City, UT (US); Kirk Loren Foote, Cottonwood Heights, UT (US)

(21) Appl. No.: 15/988,923

(22) Filed: May 24, 2018

Related U.S. Application Data
(60) Provisional application No. 62/511,132, filed on May 25, 2017.

(51) Int. Cl. A61B 46/10; A61M 5/14; A61B 46/00

(52) U.S. Cl. CPC A61B 46/10; A61B 46/40; A61M 5/1415

(57) ABSTRACT

Elongate medical appliance isolation and cleaning devices and methods are disclosed. The isolation devices provide disposable coverings of the elongate medical appliance, such as an IV pole, to protect the elongate medical appliance from contamination. The cleaning devices provide a tool to clean and disinfect the elongate medical appliance following use.

RELATED CASES

This application claims priority to U.S. Provisional Application No. 62/511,132, filed on May 25, 2017 and titled “IV POLE ISOLATION AND CLEANING DEVICES AND METHODS,” which is hereby incorporated by reference in its entirety.

### TECHNICAL FIELD

The present disclosure relates generally to medical devices. More specifically, the present disclosure relates to devices and methods to isolate and clean a medical appliance, such as an IV pole.

### BRIEF DESCRIPTION OF THE DRAWINGS

The embodiments disclosed herein will become more fully apparent from the following description and appended claims, taken in conjunction with the accompanying drawings. The drawings depict only typical embodiments, which embodiments will be described with additional specificity and detail in connection with the drawings in which:

FIG. 1A is a front view of an IV pole isolation device and an IV pole in an unassembled configuration.

FIG. 1B is a front view of the IV pole isolation device and IV pole of FIG. 1A in an assembled configuration.

FIG. 1C is a cross-sectional top view of the IV pole isolation device of FIG. 1A through sectional lines 1C-1C.

FIG. 1D is a front view of another embodiment of an IV pole isolation device and IV pole in an unassembled configuration.

FIG. 2A is a front view of another embodiment of an IV pole isolation device.

FIG. 2B is a front view of the IV pole isolation device of FIG. 2A coupled to an IV pole.

FIG. 2C is a front view of another embodiment of an IV pole isolation device coupled to an IV pole.

FIG. 3A is a cross-sectional view of a portion of an IV pole isolation device.

FIG. 3B is a front view of the IV pole isolation device of FIG. 3A.

FIG. 3C is another embodiment of an IV pole isolation device.

FIG. 4A is a perspective view of an IV pole isolation device.

FIG. 4B is a front view of the IV pole isolation device of FIG. 4A coupled to an IV pole.

FIG. 5A is a perspective view of an IV pole isolation device.

FIG. 5B is a front view of the IV pole isolation device of FIG. 5A coupled to an IV pole.

FIG. 5C is a front view of another embodiment of an IV pole isolation device coupled to an IV pole.

FIG. 6A is a perspective view of an IV pole isolation device.

FIG. 6B is a front view of the IV pole isolation device of FIG. 6A coupled to an IV pole.

FIG. 7A is a perspective view of an IV pole isolation device.

FIG. 7B is a front view of the IV pole isolation device of FIG. 7A coupled to an IV pole.

FIG. 8A is a perspective view of an IV pole cleaning device.

FIG. 8B is a front view of the IV pole cleaning device of FIG. 8A coupled to an IV pole.

FIG. 9A is a perspective view of an IV pole cleaning device.

FIG. 9B is a front view of an IV pole.

FIG. 10 is a perspective view of an IV pole cleaning device.

FIG. 11 is a front view of an IV pole cleaning device coupled to an IV pole.

FIG. 12 is a front view of another embodiment of an IV pole.

### DETAILED DESCRIPTION

Nosocomial infections are a major cause of morbidity and mortality in hospitals and other clinical settings. The source of the nosocomial infection may be a cross-contamination from a contaminated medical appliance transferred to a susceptible patient. Medical appliances, such as IV poles, are commonly utilized in hospitals and other clinical environments to support the treatment of patients. A medical appliance is commonly contaminated by body fluids from one patient, which may be transferred to a second patient. The transfer of contaminants may occur when a medical worker touches a contaminated medical appliance and then touches a patient. Prevention of nosocomial infections is a major effort of hospitals and other healthcare providers.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

As used herein, an IV pole refers to a structure, including freestanding structures and supported structures, configured to support medical devices including medicaments configured for intravenous (IV) introduction into a patient. Disclosure set forth herein relating to isolating portions of an IV pole and/or cleaning portions of an IV pole may analogously be applied to other medical structures and devices.

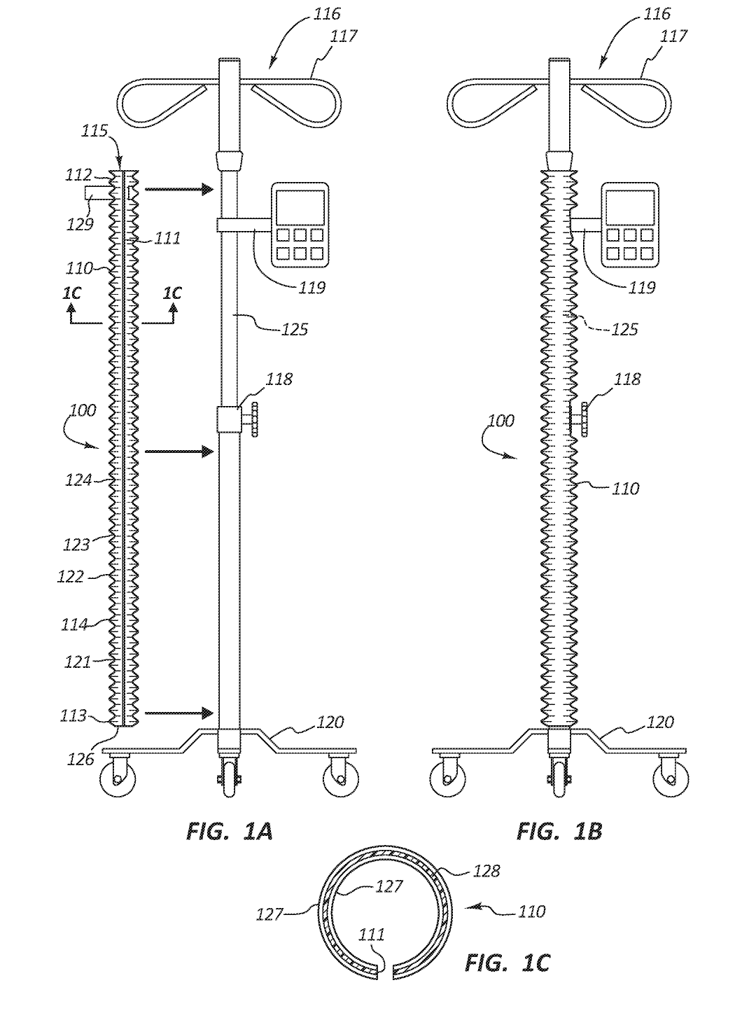

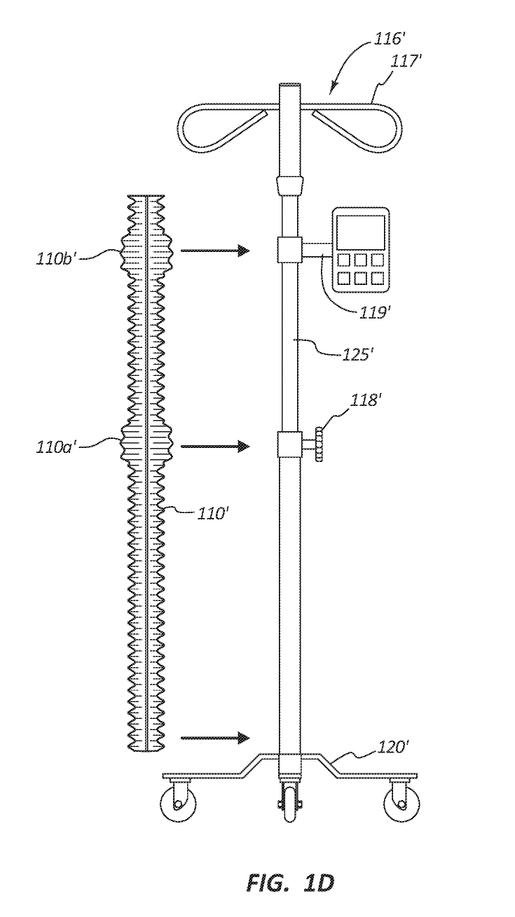

FIGS. 1A-12 illustrate different views of isolation or cleaning devices for IV poles. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

FIGS. 1A-1C are views of an embodiment of an IV pole isolation device 100. As illustrated, the isolation device 100 may comprise an elongate tube 110 comprising a bore 115, a wall 128 and a slit 111. The tube 110 may additionally comprise corrugations 122 from a first end 112 to a second end 113. The corrugations 122 may comprise a series of annular ridges 114 and annular grooves 121 alternatingly disposed along the longitudinal axis of the tube 110. In the illustrated embodiment, the corrugations 122 are configured to longitudinally collapse and extend the length of the tube 110. The tube 110 may be collapsible or extensible in a four-to-one ratio; i.e., a collapsed tube 110 may be extended to a length four times the collapsed length. When opposing longitudinal forces are applied to the first end 112 and the second end 113 of the tube 110, the tube 110 may be longitudinally lengthened. When lengthening, the ridges 114 and the grooves 121 widen, resulting in a larger separation between ridge peaks 123 and between groove bottoms 124. When a longitudinal force is applied to the first end 112 and is directed towards the second end 113, the tube 110 may be longitudinally shortened in length. When shortening, the ridges 114 and the grooves 121 narrow, resulting in a shorter separation between the ridge peaks 123 and the groove bottoms 124. The slit 111 may extend from the first end 112 to the second end 113 of the tube 110 and may be configured to facilitate longitudinal opening of the tube 110.

The tube 110 may be formed from semi-rigid material such as polypropylene, high density polyethylene, low density polyethylene, nylon, polyvinylchloride, thermoplastics, etc. The tube 110 may be opaque or translucent and may be of any suitable color for a clinical environment. The colored tube 110 may be used as an indicator of the day of the week the isolation device 100 is used. For example, a green tube 110 may indicate Monday, a blue tube 110 may indicate Tuesday, a purple tube 110 may indicate Wednesday, etc. For example, if it is Tuesday and a green isolation device 100 is in place, the healthcare worker will know that the isolation device 100 should be replaced.

In some embodiments, the isolation device 100 may comprise a time indicator 129 such that the healthcare work will be notified of a time to replace the isolation device 100 with a new isolation device 100. The time indicator 129 may be a label or a tag that is coupled to the tube 110. The time indicator 129 may be any suitable visible indicator such as a dye migration label or tag. In some embodiments, the healthcare worker would activate the time indicator 129 when initially placing the isolation device 100 over an IV pole 125. The healthcare worker would then monitor the time indicator 129 and when the time indicator 129 indicated that the isolation device 100 had been in place for a specified length of time, the healthcare worker would remove the isolation device 100 and replace it with a new isolation device 100. A time indicator, such as time indicator 129, may be utilized for any of the embodiments of an IV pole isolation device disclosed herein.

The tube 110 may comprise an antimicrobial, antifungal, and/or antiviral compound. The compound may be incorporated into the material used to form the tube 110, or the compound may be applied to the tube 110 as a coating. The compound may be in a concentration of from 1% to 4%. The compound may comprise agents such as ionic silver, zinc, copper, phenolic biocides, thiabendazole, quaternary ammonium compounds, including quaternary ammonium chlorides such as N,N-di-decyl-N,N-diethyl ammonium chloride and aromatic moieties (Benzalkonium chloride) such as N-decyl-N-benzyl-N,N-dimethylammonium chloride, photobiocidal nanoparticles including titanium dioxide, silica dioxide, and zinc dioxide, and so forth. These compounds may be utilized individually or in any combination to enhance antimicrobial properties of the tube 110. These compounds may be applied as a coating, or integrated with the underlying materials during manufacturing. The isolation device 100 may be sterilized using known techniques, such as ethylene oxide gas, gamma, e-beam, steam sterilization, hydrogen peroxide gas plasma, etc.

In some embodiments, the antimicrobial, antifungal, and/or antiviral compounds may be incorporated into an active layer, such as layer 127 of material that may be disposed on an inside and/or outside surface of the tube 110 as shown in FIG. 1C. The layer 127 may be formed from any suitable flexible or semi-flexible material, such as silicone rubber, thermoplastic elastomer, polyurethane, biodegradable materials, etc. An active layer of material comprising the antimicrobial, antifunal, and/or antivirals compounds mentioned previously may be disposed on an inside and/or outside surface of any of the embodiments of an IV pole isolation device disclosed herein. Furthermore, it is within the scope of this disclosure to position an active layer as an inner layer disposed or sandwiched between other layers. For example, an active layer, such as layer 127, may be disposed between two layers of polymeric material that allow a timed release of the active component. It is within the scope of this disclosure to utilize an inner or sandwiched active layer with any of the embodiments of an IV pole isolation device disclosed herein.

In other embodiments, the photobiocidal nanoparticles may be coated or dusted on an inside and/or outside surface of any of the embodiments of an IV pole isolation device disclosed herein or directly upon an outer surface of an IV pole. The photobiocidal nanoparticles may be activated by a handheld, whole room, or isolated UV source.

In some embodiments the tube 110 may be configured to at least partially surround an elongate medical appliance, such as an IV pole 116. The IV pole 116 may comprise an upright 125, a base 120, a height adjustor 118, an equipment clamp 119, and a bag support 117. In use, the tube 110 may be provided to a healthcare worker in a collapsed configuration. The healthcare worker may longitudinally open the tube 110 by radially outwardly expanding the tube 110 by separating opposing faces 126 of the slit 111. The healthcare worker may fit the longitudinally opened tube 110 over the upright 125 of the IV pole 116. The tube 110 may be longitudinally expanded to extend from the base 120 to the bag support 117. The slit 111 may be closed over a majority of the IV pole 116 and may be opened to accommodate components such as the height adjustor 118 and the clamp 119. Alternatively, in some embodiments, the tube 110 may be expanded to extend between IV components. For example, a first tube 110 may be disposed around the IV pole 116 and extend from the base 120 to the clamp 119, and a second tube 110 may extend from the clamp 119 to the bag support 117.

FIG. 1D illustrates another embodiment of a corrugated tube 110′ configured for use as a IV pole isolation device. As shown in the tube 110′ may be designed to couple to an IV pole 116′. In the illustrated embodiment, the IV pole 116′ comprises an upright 125′, a base 120′, a height adjuster 118′, and equipment clamp 119′, and a bag support 117′. The corrugated tube 110′ may correspond to the corrugated tuber 110 of FIGS. 1A and 1B and disclosure related in connection with that embodiment applies analogously to the embodiment of FIG. 1D.

In the embodiment of FIG. 1D, the tube 110′ is configured with larger diameter segments 110a′, 110b′ along its length. These larger diameter segments 110a′, 110b′ may be configured to accommodate portions of the IV pole 116′ to facilitate coupling of the tube 110′ to the IV pole 116′. In the illustrated embodiment, one larger diameter segment 110a′ is disposed at a longitudinal position corresponding to the height adjuster 118′ and one larger diameter segment 110b′ is disposed at a longitudinal position corresponding to the equipment clamp 119′. Embodiment with larger diameter segments at different positions, and more or fewer larger diameter segments are within the scope of this disclosure.

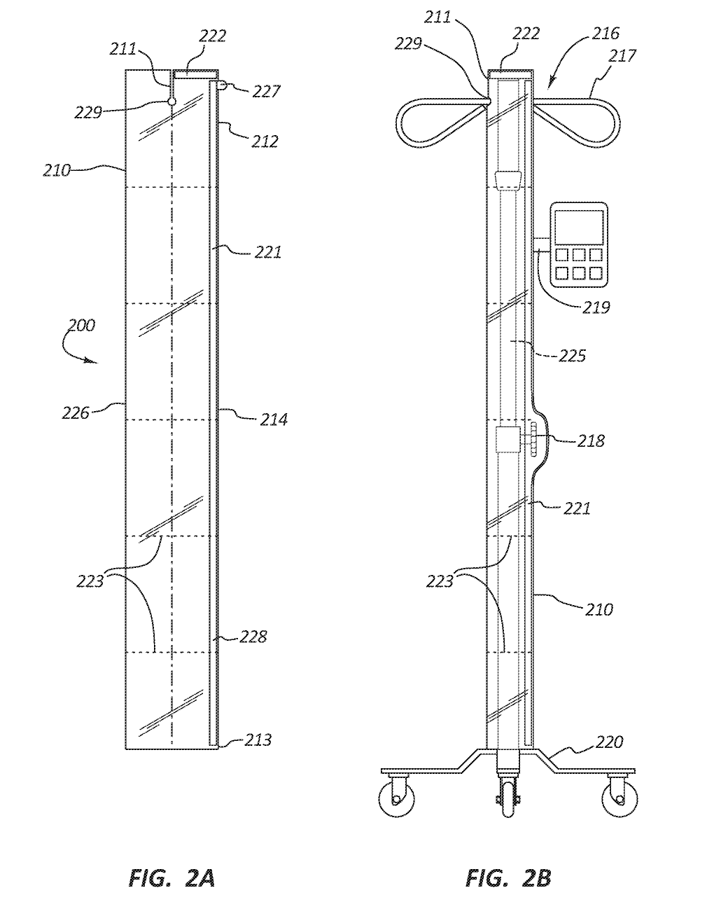

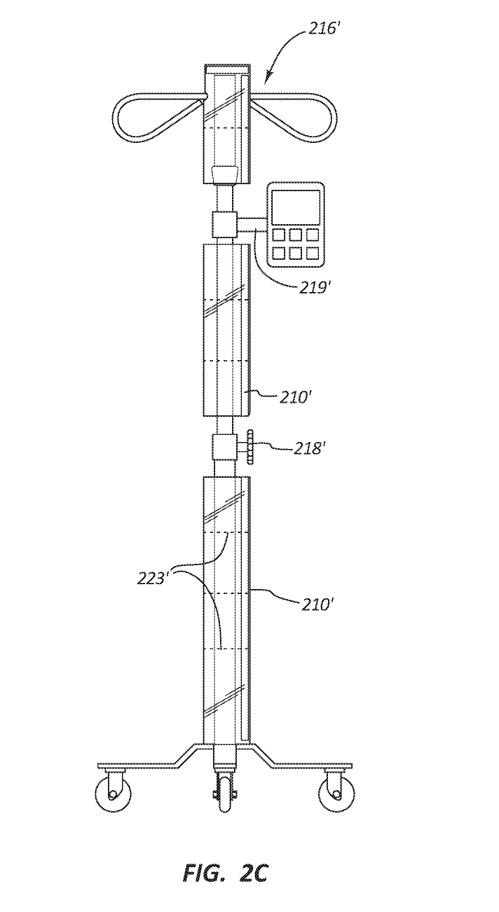

FIGS. 2A and 2B illustrate an embodiment of a medical appliance isolation device 200. The isolation device 200 comprises a rectangular sheet 210. The sheet 210 may comprise a longitudinal adhesive strip 221, a transverse adhesive strip 222, and a slit 211. The sheet 210 may be formed of a plastic material such as polypropylene or polyethylene, or any other suitable material. The sheet 210 may be opaque or translucent and may be of any suitable color for a clinical environment. The colored sheet 210 may be used as an indicator of the day of the week the isolation device 200 is used. For example, a green sheet 210 may indicate Monday, a blue sheet 210 may indicate Tuesday, a purple sheet 210 may indicate Wednesday, etc. For example, if it is Tuesday and a green isolation device 200 is in place on an IV pole 216, the healthcare worker will know that the isolation device 200 should be replaced.

The sheet 210 may comprise an antimicrobial, antifungal, and/or antiviral compound. The compound may be incorporated into the material used to form the sheet 210, or the compound may be applied to the sheet 210 as a coating. The compound may be in a concentration of from 1% to 4%. The compound may comprise agents such as ionic silver, zinc, copper, phenolic biocides, thiabendazole, quaternary ammonium compounds, including quaternary ammonium chlorides such as N,N-di-decyl-N,N-diethyl ammonium chloride and aromatic moieties (Benzalkonium chloride) such as N-decyl-N-benzyl-N,N-dimethylammonium chloride, and so forth. These compounds may be applied as a coating, or integrated with the underlying materials during manufacturing. The isolation device 200 may be sterilized using known techniques, such as ethylene oxide gas, gamma, e-beam, steam sterilization, hydrogen peroxide gas plasma, etc.

The longitudinal adhesive strip 221 may be disposed along a first longitudinal edge 214 from a first end 212 to a second end 213 of the sheet 210 as a continuous strip. In other embodiments, the adhesive strip 221 may be disposed as segments along the edge 214. The adhesive strip 221 may comprise a double-sided adhesive film with a release liner 228. The release liner 228 may comprise a tab 227 configured to facilitate removal of the release liner 228. Alternatively, the adhesive strip 221 may be an adhesive coating applied to the sheet 210 with a release liner applied to the coating. The transverse adhesive strip 222 may be disposed at the first end 212 of the sheet 210. The adhesive strip 222 may be disposed transversely between the first longitudinal edge 214 and the slit 211. The adhesive strip 222 may be formed of like materials to the adhesive strip 221. In some embodiments, the sheet 210 may be formed from a material that provides self-adhesion or “clinging,” analogous to food plastic wrap. The sheet 210 may be formed of materials such as biaxially oriented polypropylene, polyvinyl chloride, polyvinylidene chloride, and linear low density polyethylene. At least one slit 211 may be disposed at the first end 212. The slit may extend longitudinally from the first end 212 toward the second end 213 and comprise a circular hole 229 at the end of the slit 211. The sheet 210 may further comprise transverse perforations 223. The perforations 223 may be spaced at intervals of from six inches to one foot from the first end 212 to the second end 213 of the sheet 210. The perforations 223 may be configured to shorten the sheet 210 to a desirable length.

In some embodiments the medical appliance isolation device 200 may be configured to at least partially surround an elongate medical appliance, such as the IV pole 216. The IV pole 216 may comprise an upright 225, a base 220, a height adjustor 218, an equipment clamp 219, and a bag support 217. In use, the healthcare worker may place the sheet 210 over the IV pole 216 with at least one arm of the bag support 217 disposed through the slit 211 and the first end 212 extending above the IV pole 216. The healthcare worker may remove the release liner 228 of the transverse adhesive strip 222 and adhere the opposing surface 224 of the sheet 210 to the adhesive strip 222. The healthcare worker may remove the release liner 228 from the longitudinal adhesive strip 221 and adhere a second longitudinal edge 226 to the first longitudinal edge 214. The sheet 210 may extend from the bag support 217 to the base 220 of the IV pole 216.

In the embodiment of FIG. 2B, the sheet 210 extends over the height adjuster 218. The sheet 210 may extend over the height adjuster with sufficient slack that a healthcare worker may gasp the height adjuster 218 and a portion of the sheet 210 simultaneously to manipulate the height adjuster 218. Similarly, in some embodiment an adjustment portion of other components, such as the clamp 219 may be disposed within the sheet 210 with sufficient slack to allow manipulation. In some embodiments, the clamp 219 and/or the height adjustor 218 may extend outside of the closed sheet 210 (such as through an aperture or slit) as the sheet 210 extends past the clamp 219 and/or adjustor 218. The sheet 210 may be closed above and below the clamp 219 and/or adjustor 218.

The healthcare worker may remove excess length of the sheet 210 by transversely tearing the sheet 210 at one of the perforations 223. In other embodiments, the healthcare worker may wrap the sheet 210 around the IV pole 216 such that the sheet 210 clings to the upright 225 or to itself.

FIG. 2C is a front view of an IV pole isolation device coupled to an IV pole 216′. Analogous to the embodiment of FIGS. 2A and 2B, the IV pole isolation device of FIG. 2C comprises sheets 210′ configured to be coupled to, and isolate, the IV pole 216′. Disclosure recited in connection with the embodiment of FIGS. 2A and 2B analogously applies to the embodiment of FIG. 2C.

In the embodiment of FIG. 2C, the sheet 210′ is applied in segments over the IV pole 216′, such as between the height adjustor 218′ and the clamp 219′. A healthcare worker may create openings for various components by simply separating the sheet 210′ at one or more perforations 223′ and apply the sheet 210′ in segments along the IV pole 216′.

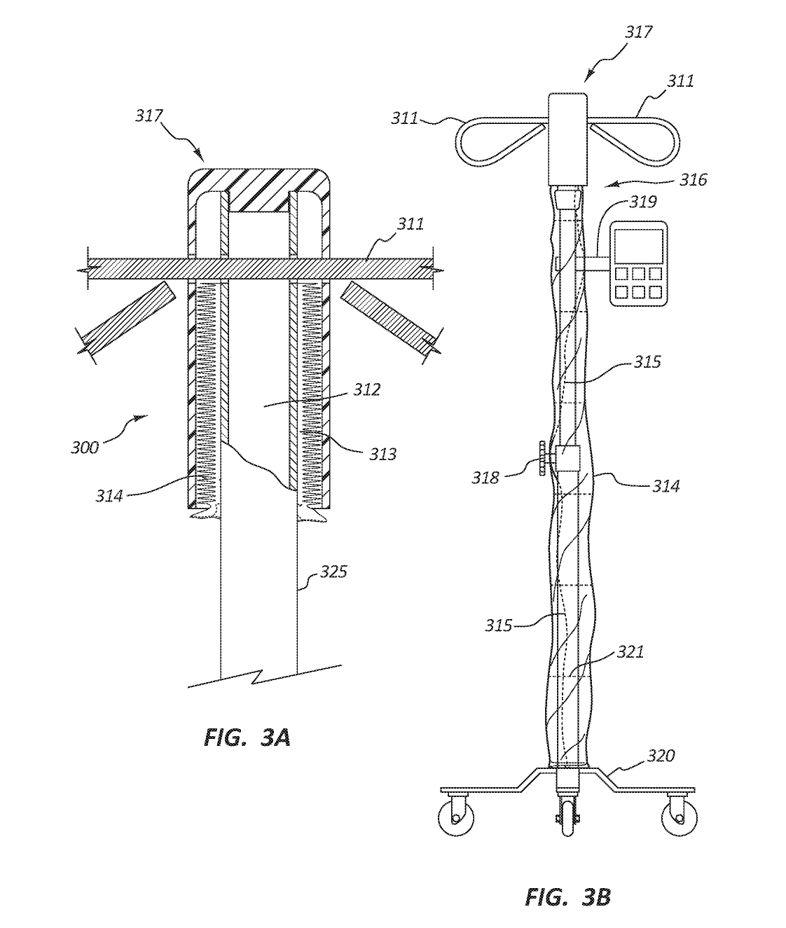

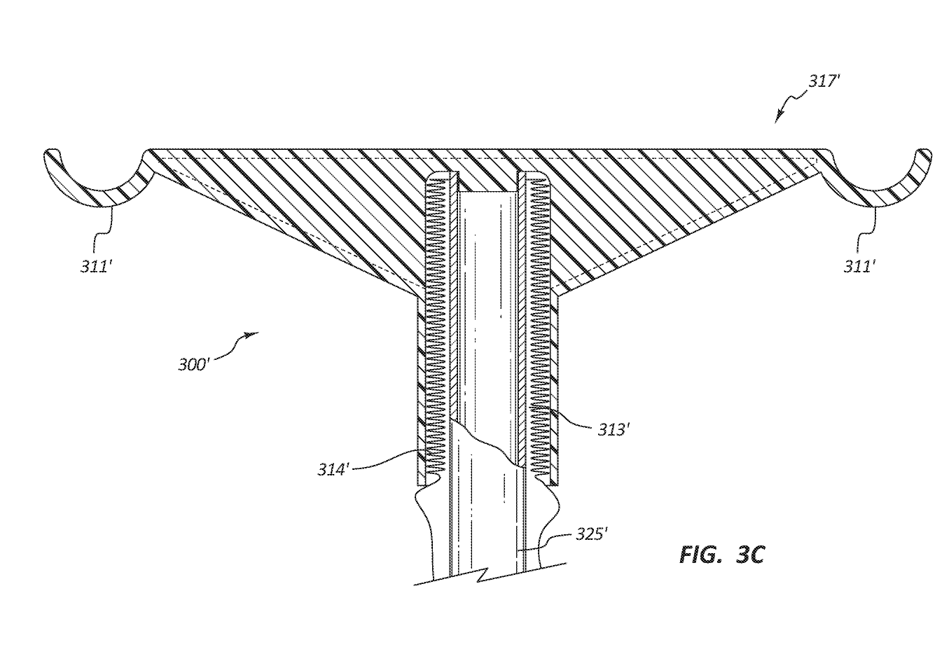

FIGS. 3A and 3B illustrate an embodiment of a medical appliance isolation device 300, with FIG. 3A being a cross-sectional view of a portion of the IV pole isolation device 300. The isolation device 300 may comprise a bag support 317 and an isolation sheath or sleeve 314. The isolation device 300 may be a single-use or multi-use device. The bag support 317 may be configured with one, two, or more hooks 311 extending radially outward from a central cavity 312. The hooks 311 may be configured to hang and support a bag or bottle of intravenous fluid or medication. The central cavity 312 may be configured to couple the bag support 317 to an end of an upright 325 of an IV pole 316. An annular cavity 313 may surround the central cavity 312. The annular cavity 313 may retain a longitudinally compressed isolation sheath 314. The isolation sheath 314 may be formed of a plastic material such as polypropylene or polyethylene, or any other suitable material. The sheath 314 may be opaque or translucent and may be of any suitable color for the clinical environment. The colored sheath 314 may be used as an indicator of the day of the week the isolation device 300 is used. For example, a green sheath 314 may indicate Monday, a blue sheath 314 may indicate Tuesday, a purple sheath 314 may indicate Wednesday, etc. For example, if it is Tuesday and a green isolation device 300 is in place, the healthcare worker will know that the isolation device 300 should be replaced.

The sheath 314 may comprise an antimicrobial, antifungal, and/or antiviral compound. The compound may be incorporated into the material used to form the sheath 314, or the compound may be applied to the sheath 314 as a coating. The compound may be in a concentration of from 1% to 4%. The compound may comprise agents such as ionic silver, zinc, copper, phenolic biocides, thiabendazole, quaternary ammonium compounds, including quaternary ammonium chlorides such as N,N-di-decyl-N,N-diethyl ammonium chloride and aromatic moieties (Benzalkonium chloride) such as N-decyl-N-benzyl-N,N-dimethylammonium chloride, and so forth. These compounds may be applied as a coating, or integrated with the underlying materials during manufacturing. The isolation device 300 may be sterilized using known techniques, such as ethylene oxide gas, gamma, e-beam, steam sterilization, hydrogen peroxide gas plasma, etc. The sheath 314 may further comprise annular perforations 321 spaced at intervals of six inches or 12 inches along the length of the sheath 314. The sheath 314 may be detachable from the bag support 317 such that a new sheath 314 may be attached.

In some embodiments, the medical appliance isolation device 300 may be configured to at least partially surround an elongate medical appliance, such as the IV pole 316. The IV pole 316 may comprise the upright 325, a base 320, and a height adjustor 318. In use, a healthcare worker may position the bag support 317 over a free end of the upright 325 of the IV pole 316. The healthcare worker may extend the isolation sheath 314 from the annular cavity 313 and over the upright 325 and the height adjustor 318 to the base 320. Unwanted length of the sheath 314 may be removed by tearing the sheath 314 at one of the annular perforations 321.

The bag support member 317 may comprise a standard bag support such as those routinely coupled to an IV pole and the isolation device 300 may be configured to fit over the bag support member 317. In some embodiments the isolation device 300 may comprise a molded part configured to fit over and isolate the bag support member 317 as well as other portions of the IV pole 316.

In other embodiments, such as shown in FIG. 3C, the bag support member 317′ may be an integrally molded portion of the isolation device 300′. For example, an isolation device 300′ may comprise a bag support member 317′ molded together with a housing comprising an annular cavity 313′ which contains an isolation sheath 314′. The bag support member 317′ may comprise one or more hooks 311′ for coupling with components such as IV bags. In such embodiments, the isolation device 300′ may be placed on the upright 325′ of an IV pole in place of a standard bag support member. The isolation device 300′, including the integrated bag support member 317′ and sheath 314′ may be configured for one time use, meaning the entire isolation device 300′ is replaced between uses of the IV pole.

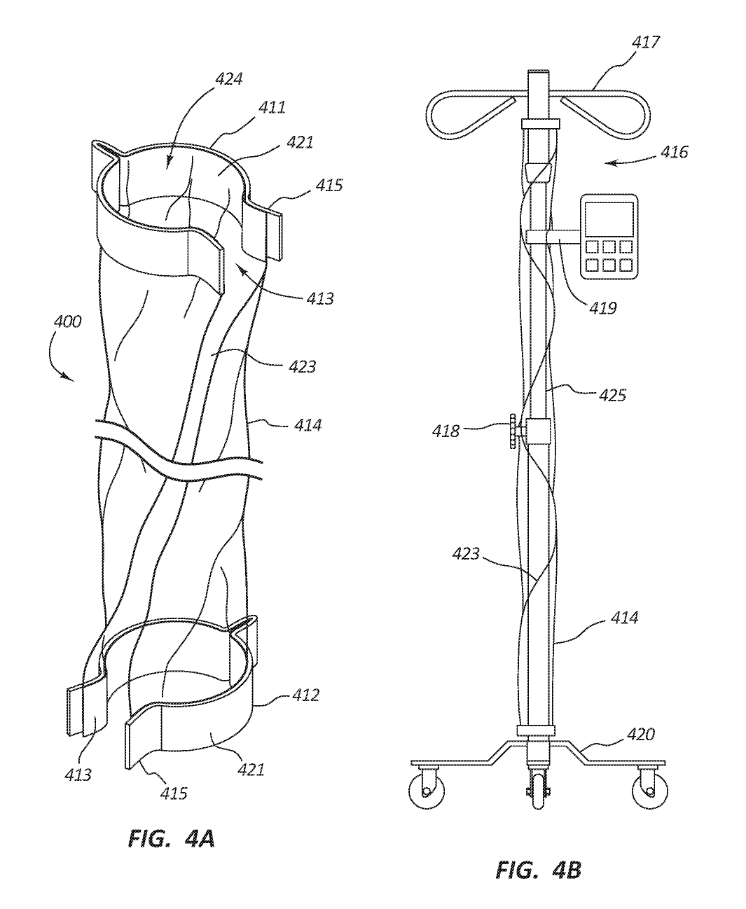

FIGS. 4A and 4B illustrate an IV pole isolation device 400. The isolation device 400 may comprise a first clamp 411, a sheath or sleeve 414, and a second clamp 412. The first clamp 411 and the second clamp 412 may be “C” shaped with an opening 413 on one side. Lever arms 415 may extend from the clamps 411, 412. The clamps 411, 412 may be configured to slide over and frictionally couple to an upright 425 of an IV pole 416. In some embodiments, the clamps 411, 412 may comprise a magnetic material and be configured to couple to the upright 425 with a magnetic force. The clamps 411, 412 may be formed from a plastic or metal material, such as polyethylene, polypropylene, polycarbonate, stainless steel, aluminum, etc.

The sheath 414 may be fixedly coupled to the clamps 411, 412 such that the clamp opening 413 is oriented with a longitudinal opening 423 of the sheath 414. The sheath 414 may be coupled to a clamp inside wall 421 or an outside wall using any suitable technique, such as welding, gluing, overmolding, etc. In some embodiments, additional clamps (not shown) similar to clamps 411, 412 may be provided such that the additional clamps 426 may be coupled to the sheath 414 and the upright 425 following application of the sheath 414 to the IV pole 416. The sheath 414 may be longitudinally compressed such that the sheath 414 is contained within a bore 424 of the first clamp 411 and the second clamp 412.

In some embodiments, the sheath 414 of isolation device 400 may be formed of a plastic material such as polypropylene or polyethylene, or any other suitable material. The sheath 414 may be opaque or translucent and may be of any suitable color for the clinical environment. The colored sheath 414 may be used as an indicator of the day of the week the isolation device 400 is used. For example, a green sheath 414 may indicate Monday, a blue sheath 414 may indicate Tuesday, a purple sheath 414 may indicate Wednesday, etc. For example, if it is Tuesday and a green isolation device 400 is in place, the healthcare worker will know that the isolation device 400 should be replaced.

The sheath 414 may comprise an antimicrobial, antifungal, and/or antiviral compound. The compound may be incorporated into the material used to form the sheath 414, or the compound may be applied to the sheath 414 as a coating. The compound may be in a concentration of from 1% to 4%. The compound may comprise agents such as ionic silver, zinc, copper, phenolic biocides, thiabendazole, quaternary ammonium compounds, including quaternary ammonium chlorides such as N,N-di-decyl-N,N-diethyl ammonium chloride and aromatic moieties (Benzalkonium chloride) such as N-decyl-N-benzyl-N,N-dimethylammonium chloride, and so forth. These compounds may be applied as a coating, or integrated with the underlying materials during manufacturing. The isolation device 400 may be sterilized using known techniques, such as ethylene oxide gas, gamma, e-beam, steam sterilization, hydrogen peroxide gas plasma, etc.

In some embodiments, the medical appliance isolation device 400 may be configured to at least partially surround an elongate medical appliance, such as the IV pole 416. The IV pole 416 may comprise the upright 425, a base 420, a bag support 417 and a height adjustor 418. In use, a healthcare worker may couple the first clamp 411 to the upright 425 of the IV pole 416 just below the bag support 417. The sheath 414 is extended to the base 420 of the IV pole 416. The sheath 414 is disposed around the upright 425 through the sheath opening 423. The second clamp 412 is coupled to the upright 425. Either the first clamp 411 or the second clamp 412 is rotated around the upright 425 while the other one is held stationary, or the first clamp 411 is rotated in one direction and the second clamp 412 is rotated in a second direction. Rotation of the clamps 411, 412 may be accomplished by applying a rotational force to the lever arms 415. Rotation of the clamps 411, 412 results in closing of the sheath opening 423 and conforming of the sheath 414 to the shape of the upright 425 in a spiral configuration. The sheath opening 423 may remain open as the sheath 414 passes around components of the IV pole 416 that are too large to be covered by the sheath 414, such as the equipment clamp 419 and the height adjustor 418. Additional clamps 426 may be coupled to the sheath 414 adjacent to the equipment clamp 419. The additional clamps 426 may be rotated to close portions of the sheath 414 between components of the IV pole 416.

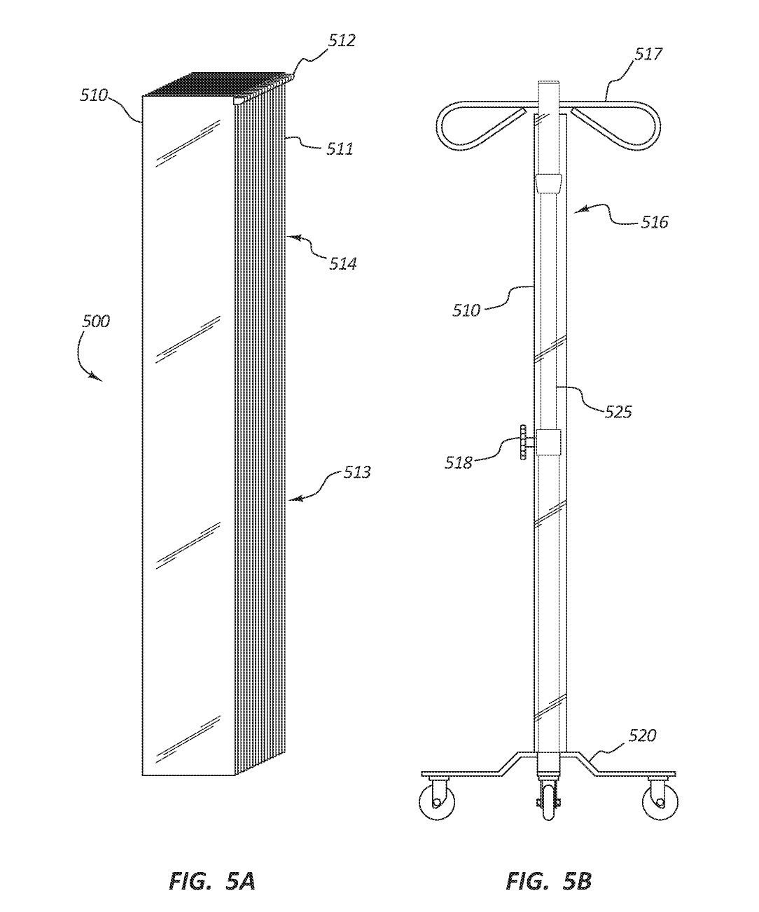

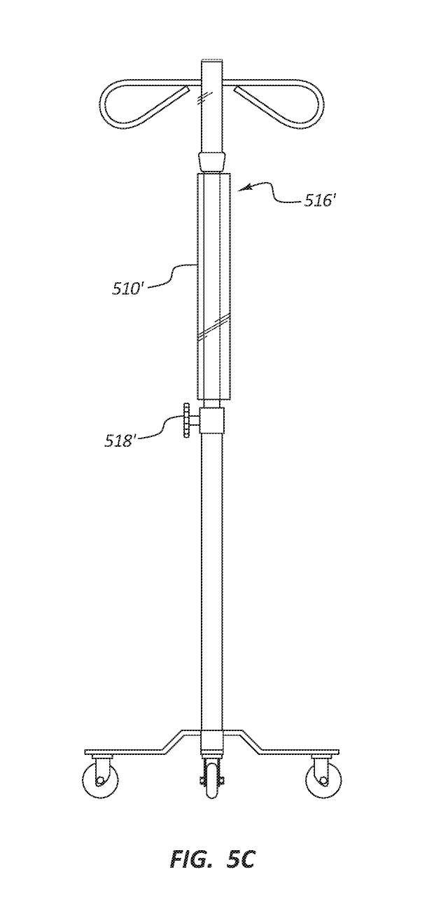

FIGS. 5A and 5B illustrate an embodiment of an IV pole isolation device 500. The isolation device 500 may comprise a sheet 510 configured to at least partially surround an upright 525 of an IV pole 516. The sheet 510 may comprise multiple film layers 511 stacked upon one another. In other embodiments the layers 511 may be coupled together along a perforated longitudinal edge and rolled forming a series of film layers separable at the perforated edge. The sheet 510 may be rectangular in shape and include 10 to 20 film layers 511. The film layers 511 may be formed of a plastic material such as polypropylene or polyethylene, or any other suitable material. The film layers 511 may be opaque or translucent and may be of any suitable color for a clinical environment. The colored film layers 511 may be used as an indicator of the day of the week a film layer 511 was used. For example, a green film layer 511 may indicate Monday, a blue film layer 511 may indicate Tuesday, a purple film layer 511 may indicate Wednesday, etc. Thus, if it is Tuesday and a green film layer 511 is in place, the healthcare worker will know that the film layer 511 should be removed.

The film layers 511 may comprise an antimicrobial, antifungal, and/or antiviral compound. The compound may be incorporated into the material used to form the film layers 511, or the compound may be applied to the film layers 511 as a coating. The compound may be in a concentration of from 1% to 4%. The compound may comprise agents such as ionic silver, zinc, copper, phenolic biocides, thiabendazole, quaternary ammonium compounds, including quaternary ammonium chlorides such as N,N-di-decyl-N,N-diethyl ammonium chloride and aromatic moieties (Benzalkonium chloride) such as N-decyl-N-benzyl-N,N-dimethylammonium chloride, and so forth. These compounds may be applied as a coating, or integrated with the underlying materials during manufacturing. The isolation device 500 may be sterilized using known techniques, such as ethylene oxide gas, gamma, e-beam, steam sterilization, hydrogen peroxide gas plasma, etc.

Each film layer 511 may comprise an adhesive backing 513. The adhesive backing 513 may completely cover the back surface 514 of the film layer 511. In some embodiments, the adhesive backing 513 is disposed to partially cover the back surface 514. The adhesive backing 513 may be disposed as dots, stripes, dashes, etc. In other embodiments, each film layer 511 is self-adhering. The film layer 511 may be formed from a material that provides self-adhesion or “clinging,” analogous to food plastic wrap. The film layer 511 may be formed of materials such as biaxially oriented polypropylene, polyvinyl chloride, polyvinylidene chloride, and linear low density polyethylene. Each film layer 511 may further comprise an indicium tab 512 coupled to one corner of the film layer 511. The tabs 512 may be marked with sequential numbers or letters indicating the sequence position of a particular film layer 511. For example, if the tab 512 is marked with a “5,” there are five film layers 511 remaining in the sheet 510.

In some embodiments, the medical appliance isolation device 500 may be configured to at least partially surround an elongate medical appliance, such as the IV pole 516. The IV pole 516 may comprise the upright 525, a base 520, a bag support 517, and a height adjustor 518. In use, a healthcare worker may apply the isolation device 500 onto the upright 525 between the bag support 517 and the base 520 such that the sheet 510 at least partially surrounds and conforms to the shape of the upright 525. When a clean film layer 511 is desired, the healthcare worker may grasp the tab 512 and pull a dirty film layer 511 from the sheet 510, exposing a new film layer 511 and clean surface. The process may be repeated until the final film layer 511 is removed. The tab 512 of the final film layer 511 may be marked with a “1” indicating that it is the final film layer 511.

FIG. 5C is a front view of another embodiment of an IV pole isolation device comprising a multiple film layer sheet 510′ coupled to an IV pole 516′. Disclosure recited in connection with the sheet 510 of FIGS. 5A and 5B applies analogously to the sheet 510′ of FIG. 5C.

In the embodiment of FIG. 5C, the sheet 510′ is disposed along only a portion of the IV pole 516′, in this case above the height adjustor 518′. In other embodiments, it may be disposed along different portions. The sheet 510′ may be disposed along only the portion of the IV pole 516′ which is generally grasped by a user. This may facilitate coupling of the sheet 510′ to the IV pole 516′ without interference from structures along the IV pole 516′, while still providing a clean gripping surface (and removable layers) along a portion of the IV pole 516′.

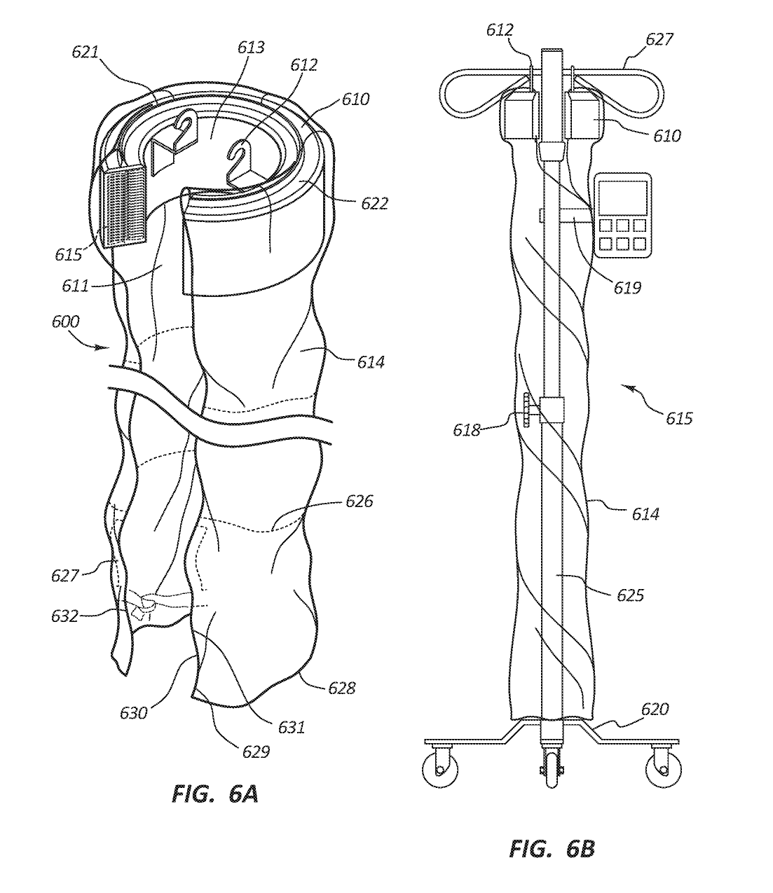

FIGS. 6A and 6B illustrate an embodiment of an IV pole isolation device 600. The isolation device 600 may comprise a dispenser 610 and a sheath or sleeve 614. The dispenser 610 may be configured as a circular ring shape with a central bore 613 sized to fit around an upright 625 of an IV pole 616. An annular cavity 615 is located within the dispenser 610. An opening 621 of the cavity 615 may be located at a top surface 622 of the dispenser 610. The dispenser 610 may comprise a notch 611 such that the ring shape is not continuous. In other embodiments, the dispenser 610 may form a continuous ring shape. The notch 611 may be configured such that the upright 625 passes through the notch 611 into the central bore 613. The dispenser 610 may further comprise a support bracket 612 configured to couple the dispenser 610 to a bag support 617 or a base 620 of the IV pole 616. The dispenser 610 may be formed from any suitable semi-rigid plastic material, such as polypropylene or polyethylene, using suitable manufacturing techniques, such as injection molding.

In some embodiments, the sheath 614 is disposed within the cavity 615 in a longitudinally compressed configuration. The sheath 614 may be formed of a plastic material such as polypropylene or polyethylene, or any other suitable material. The sheath 614 may be opaque or translucent and may be of any suitable color for a clinical environment. The coloring of the sheath 614 may be in sequential lengths. For example, a first length of the sheath 614—adequate to cover the upright 625 of the IV pole 616—may be green; a second length may be blue; a third length may be purple; etc. The colored sheath 614 may be used as an indicator of the day of the week the sheath 614 is used. For example, if it is Tuesday and a green sheath 614 is in place, the healthcare worker will know that the sheath 614 section should be replaced. The sheath 614 may comprise an antimicrobial, antifungal, and/or antiviral compound. The compound may be incorporated into the material used to form the sheath 614, or the compound may be applied to the sheath 614 as a coating. The compound may comprise agents such as ionic silver, zinc, copper, phenolic biocides, thiabendazole, quaternary ammonium compounds, including quaternary ammonium chlorides such as N,N-di-decyl-N,N-diethyl ammonium chloride and aromatic moieties (Benzalkonium chloride) such as N-decyl-N-benzyl-N,N-dimethylammonium chloride, and so forth. These compounds may be applied as a coating, or integrated with the underlying materials during manufacturing. The sheath 614 may be configured with a longitudinal opening 624 in alignment with the notch 611. Ties 627 may extend into the opening 624 and may be partially detachable from the sheath 614. Transverse perforations 626 may be spaced six inches to 12 inches along the longitudinal axis of the sheath 614. The sheath 614 may comprise an adhesive strip 629 along a first longitudinal edge 630. The adhesive strip 629 may be continuous along the first edge 630, or the strip 629 may be segmented. For example, the segments may correspond to the transverse perforations 626.

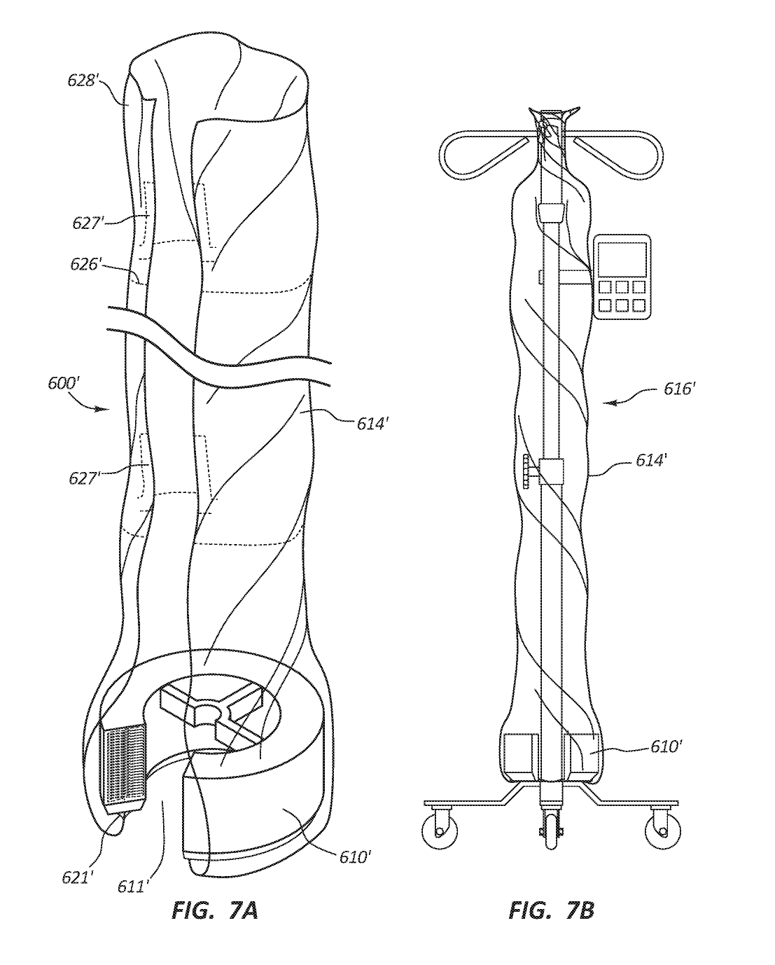

In some embodiments, the isolation device 600 may be configured to at least partially surround an elongate medical appliance, such as the IV pole 616. The IV pole 616 may comprise the upright 625, the base 620, the bag support 617, a height adjustor 618, and an equipment clamp 619. In use, the healthcare worker may position the dispenser 610 adjacent to the bag support 617 or the base 620 of the IV pole 616. The dispenser 610 may be coupled to the IV pole 616 utilizing the bracket 612. When the dispenser 610 is positioned adjacent the bag support 617, the cavity opening 621 is oriented upwards. (In embodiments wherein the dispenser is positioned adjacent the base, the cavity opening 621 is oriented downwards, such as described in connection with FIGS. 7A and 7B). A free end 628 of the sheath 614 is pulled from the cavity 615, and the sheath 614 is draped over the dispenser 610, such that the dispenser is within the sheath 614 when the sheath 614 is deployed. In other words as shown in FIGS. 6A and 6B, the free end 628 of the sheath 614 is pull upwards from the cavity opening 621 and then downwards such that the dispenser 610 is covered by the sheath 614. The sheath 614 is extended to cover the upright 625. The sheath 614 may partially cover the height adjustor 618 and/or the equipment clamp 619. The length of the sheath 614 may be adjusted by tearing the sheath 614 at one of the transverse perforations 626. The sheath opening 624 is closed by removal of a release liner 631 from the adhesive strip 629 and adhering the first longitudinal edge 630 to a second longitudinal edge 632. Portions of the release liner 631 may remain in place around the equipment clamp 619 and/or the height adjustor 618. The free end of the sheath 614 may be coupled to the bag support 617, the base 620, or the upright 625 using the ties 627. Alternatively, the free end 628 may be coupled to the upright 625 utilizing any suitable technique, such as a clamp, tape, etc. In some embodiments, the ties 627 may comprise an adhesive layer covered by a release liner. In other embodiments, the ties 627 may comprise teeth disposed on outer edges of the ties 627. The sheath 614 can have vertical slits corresponding to locations of the ties 627 and configured to engage the teeth of the ties 627 such that the ties 627 can cinch the sheath 614 tightly around the IV pole 616.

FIGS. 7A and 7B illustrate another embodiment of an IV pole isolation device 600′, which has analogous features to the IV pole isolate device of FIGS. 6A and 6B. Like components are designated with like numerals and disclosure recited in connection with FIGS. 6A and 6B applies to the embodiment of FIGS. 7A and 7B. In the embodiment of FIGS. 7A and 7B, the dispenser 610′ is disposed at the bottom of the upright 625′ of the IV pole 616′. As with the embodiment of FIGS. 6A and 6B, a dispenser ring 610′ and a sheath or sleeve 614′ may be circular and continuous or comprise a notch 611′.

The sheath 614′ may be pulled up from the bottom of the IV pole 616′, around the dispenser 610′ and toward the top of the IV pole 616′. Thus, the sheath 614′ may enclose the dispenser 610′. In other words as shown in FIGS. 7A and 7B, in some configurations, the free end 628′ of the sheath 614′ is pulled downwards from the cavity opening 621′ and then upwards such that the dispenser 610 is covered by the sheath 614. Further, the sheath 614′ may be manipulated to accommodate structures on the IV pole 616′ and to surround portions of the IV pole 616′ and associated medical equipment. Adhesive strips 629′, ties 627′, and transverse perforations 626′ may be configured to facilitate coupling and sizing of the sheath 614′.

With respect to the embodiments of IV pole isolation devices disclosed in connection with FIGS. 1A-7B, any of these IV pole isolation devices may be configured to cover a portion of an IV pole, or may be modified to cover the entire IV pole. In other words, these IV pole isolation devices may be configured to at least partially cover an IV pole. Still further, these IV pole isolation devices may be configured to discontinuously cover portions of an IV pole, while leaving intermediate portions exposed.

Additionally, the isolation cover materials discussed with any of the embodiments above may be configured to conform to the IV pole and/or components attached to the IV pole.

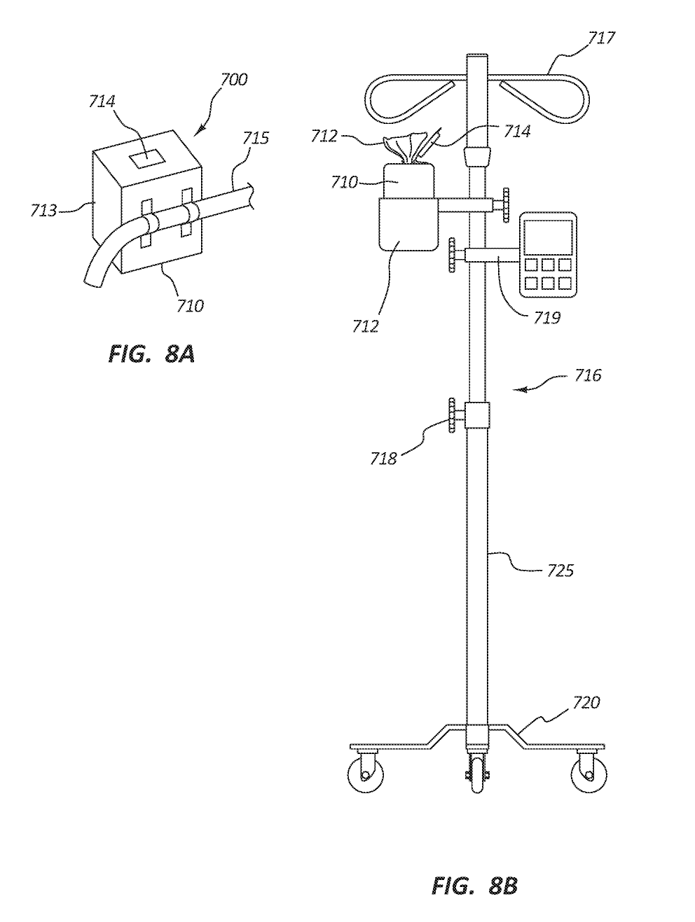

FIGS. 8A and 8B illustrate an embodiment of an IV pole cleaning device 700. The cleaning device 700 may comprise a dispenser 710 and disinfecting wipes 712. The dispenser 710 may be configured as a generally rectangular container 713 with a sealable lid 714. The container 713 may comprise straps formed from hook-and-loop fasteners and configured to secure the container 713 to an IV pole 716. In some embodiments, the container 713 may comprise a bracket (not shown) configured to couple to an equipment clamp 719 of the IV pole 716. In other embodiments, the container 713 may be retained within a holder 722 coupled to the IV pole 716. The container 713 may retain the disinfecting wipes 712. The disinfecting wipes may be formed from any natural or synthetic suitable material, such as cotton, polyester, polypropylene, foam, etc. The wipes 712 may be saturated with any suitable cleaning or disinfecting solution.

In some embodiments, the IV pole cleaning device 700 may be configured to facilitate cleaning or disinfecting of the IV pole 716. The IV pole 716 may comprise an upright 725, a base 720, a bag support 717, a height adjustor 718, and the equipment clamp 719. In use, the healthcare worker may couple the container 713 to the upright 725. The lid 714 is displaced and a wipe 712 is removed from the container 713. All components of the IV pole 716, including the upright 725, the base 720, the bag support 717, the height adjustor 718, and the equipment clamp 719, are wiped with the cleaning or disinfecting solution of the wipe 712.

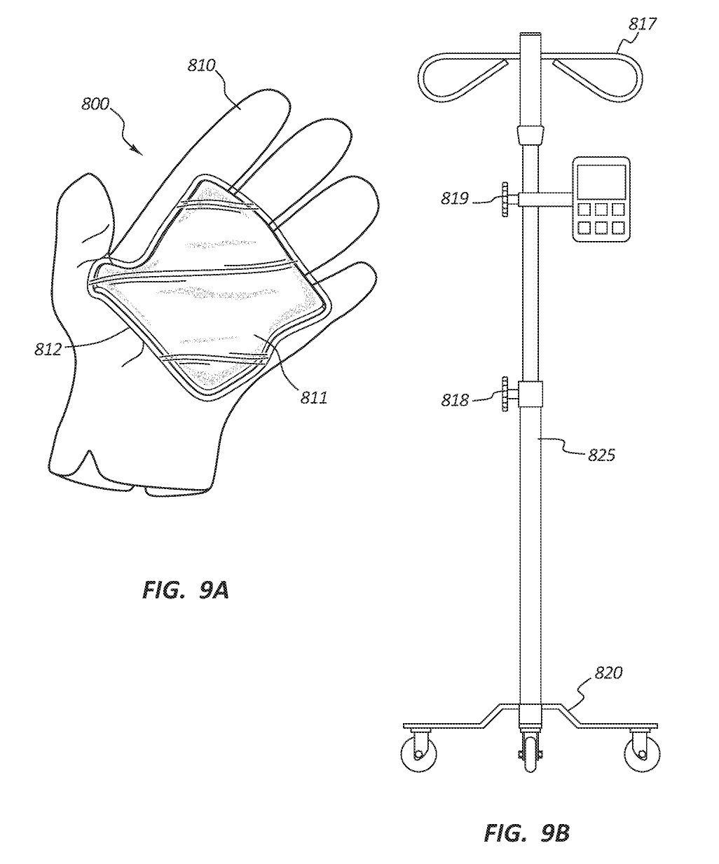

FIGS. 9A and 9B illustrate an embodiment of an IV pole cleaning device 800. The cleaning device 800 comprises a glove 810 and an absorbent pad 811. The glove 810 is configured to fit over the hand of the healthcare worker. The glove 810 may be formed from any material suitable for a medical environment, such as nitrile rubber, polyvinylchloride, neoprene rubber, etc. The glove 810 may be configured to be monodextrous; i.e., it may be configured to be either a left-handed glove or a right-handed glove. The glove 810 may be configured such that one glove 810 fits over hands of various sizes, or the glove 810 may be configured to fit a specific-size hand, such as small, medium, large, etc. The absorbent pad 811 is coupled to the glove 810. The pad 811 may be formed from any natural or synthetic material suitable for the clinical environment, such as cotton, polypropylene, polyester, foam, etc.

The pad 811 may be coupled to the glove 810 using any suitable technique, such as adhesive, glue, welding, etc. The pad 811 may be disposed on the palm area of the glove 810 or, alternatively, the pad 811 may be disposed on the palm area and the fingers. In some embodiments, the pad 811 may comprise a cleaning or disinfecting solution and a releasable seal 812. The cleaning or disinfecting solution may be retained by the pad 811. The cleaning or disinfecting solution may be any suitable solution. The releasable seal 812 may be configured to cover the pad 811 to prevent evaporation of the cleaning or disinfecting solution. The seal 812 may be configured to peel from the glove 810 prior to use of the cleaning device 800.

In some embodiments, the IV pole cleaning device 800 may be configured to facilitate cleaning or disinfecting of an IV pole 816. The IV pole 816 may comprise an upright 825, a base 820, a bag support 817, a height adjustor 818, and an equipment clamp 819. In use, the healthcare worker may select an appropriate IV pole cleaning device 800—i.e., a right-hand or left-hand configuration of the appropriate size. The glove 810 is put over the appropriate hand. The seal 812 is removed exposing the solution-saturated pad 811. All components of the IV pole 816, including the upright 825, the base 820, the bag support 817, the height adjustor 818, and the equipment clamp 819, are wiped with the pad 811. The IV pole cleaning device 800 is turned inside out and disposed of following cleaning and/or disinfecting of the IV pole 816.

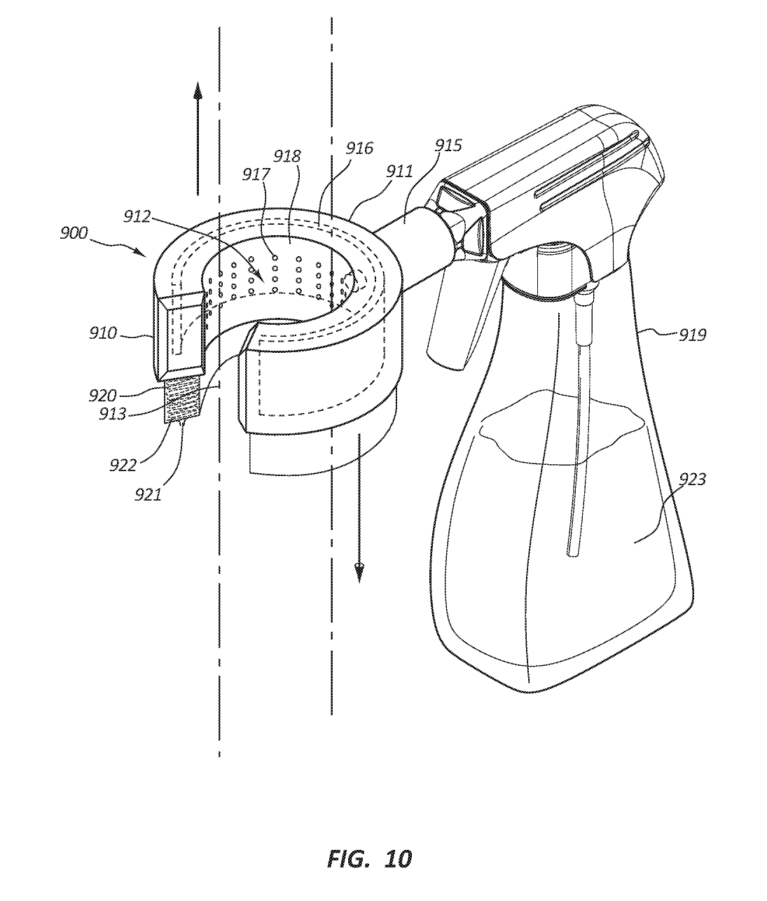

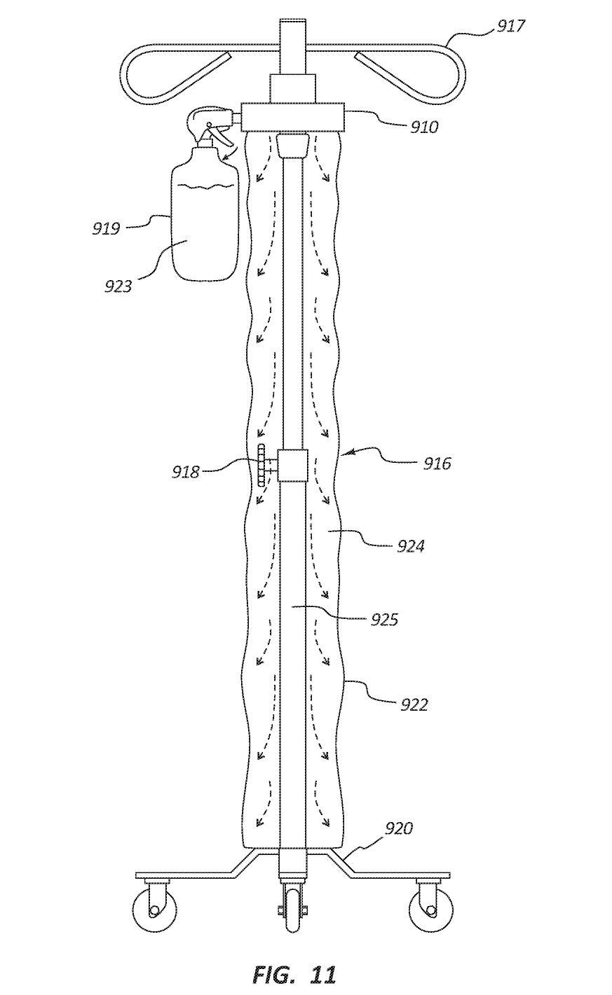

FIGS. 10 and 11 illustrate an embodiment of an IV pole cleaning device 900. The cleaning device 900 may comprise a spray ring 910. The spray ring 910 may comprise a body 911 configured as a circular ring. The body 911 may be configured as a full ring or may be configured as a discontinuous ring with a slot 913. The slot 913 may be configured to allow passage of an upright 925 of an IV pole 926 through the slot 913 and into a central bore 912. The central bore 912 may be configured to at least partially surround the upright 925. In some embodiments, the body 911 comprises a hinge (not shown) configured to allow lateral displacement of portions of the body 911 to open the slot 913 to a dimension adequate for the passage of the upright 925.

The body 911 further comprises a neck portion 915 and fluid channels 916 running from the neck into the ring-shape body 911. The fluid channels 916 may be formed directly in the body 911, or the channels 916 may comprise tubes inserted into a cavity within the body 911. The fluid channels 916 are in fluid communication with orifices 917 disposed on an inside surface 918 of the ring body 911. The number of orifices 917 is adequate to provide total annular spray coverage of the upright 925. The number of orifices 917 may range from two to 20, including from three to eight. The orifices 917 may be configured to emit a fluid spray or a mist. The neck portion 915 is coupled to a fluid source 919, such as a spray bottle having a trigger pump. The fluid channels 916 are in fluid communication with the fluid source 919.

The fluid source 919 may contain a cleaning and/or antiseptic solution 923. The solution may be tinted with a color pigment such that the healthcare worker may visually see when all of the solution 923 has been wiped from the IV pole 916. Alternatively, the solution 923 may comprise a compound that is reflective of ultraviolet light, such that the healthcare worker may shine an ultraviolet light source at an allegedly cleaned IV pole 916 and detect areas where solution remains on the IV pole 916.

Referring to FIGS. 10 and 11, in some embodiments, the body 911 of the cleaning device 900 may comprise an annular cavity 920 extending from one side of the body 911. The cavity 920 has an opening 921. A sheath or sleeve 922 is longitudinally compressed in the cavity 920. The sheath 922 may be formed from any suitable material, such as polyethylene, polypropylene, etc.

In some embodiments, the IV pole cleaning device 900 may be configured to facilitate cleaning or disinfecting of the IV pole 926. The IV pole 916 may comprise the upright 925, a base 927 a bag support 928, a height adjustor 929, and an equipment clamp 930. In use, the healthcare worker may couple the cleaning device 900 to the fluid source 919 containing a cleaning and/or antiseptic solution 923. The spray ring 910 of the cleaning device 900 is fitted over the upright 925 such that the spray ring 910 at least partially surrounds the upright 925. The solution 923 is forced into the fluid channels 916 and through the orifices 917. The solution 923 may exit the orifices 917 as a spray or a mist. The cleaning device 900 is displaced over the longitudinal axis of the upright 925 such that the upright 925 is coated with the solution 923. The cleaning device 900 is removed from surrounding the upright 925, and the upright 925 is wiped with a cloth.

In other embodiments, after the spray ring 910 of the cleaning device 900 is fitted over the upright 925, the sheath 922 is extended from the cavity 920 of the spray ring body 911 over the upright 925 such that a chamber 924 is formed by the sheath 922 around the upright 925. The solution 923 is forced into the fluid channels 916 and through the orifices 917. The solution 923 may exit the orifices 917 as a mist to fill the chamber 924. The cleaning device 900 is held in place until the mist coats the upright 925. The cleaning device 900 is removed from surrounding the upright 925, and the upright 925 is wiped with a cloth.

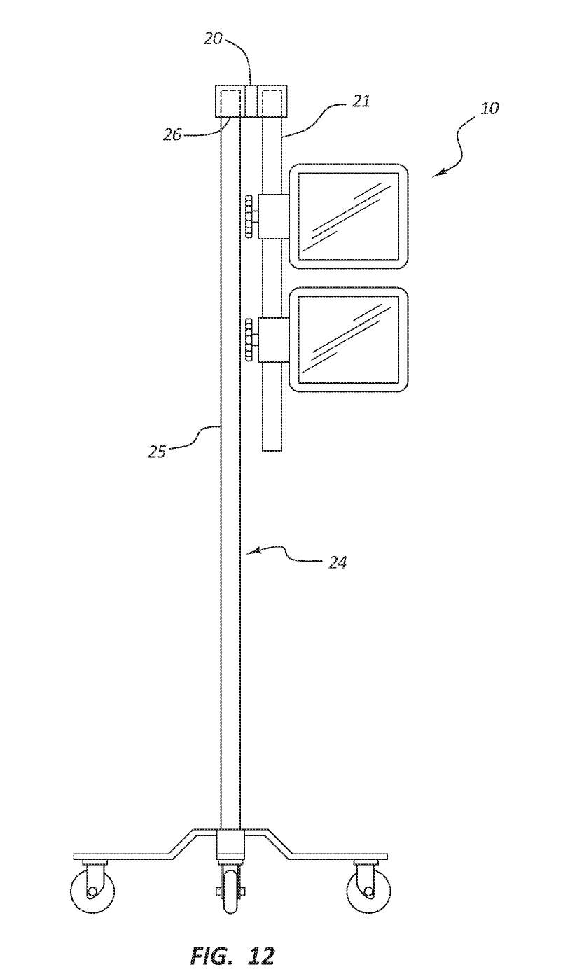

FIG. 12 illustrates an embodiment of an auxiliary IV pole 10. The auxiliary pole 10 may be configured such that medical equipment, such as IV pumps, may be mounted to the auxiliary pole 10, leaving an IV pole 24 free of mounted medical equipment such that an isolation device, as described previously, may cover the IV pole 24 without obstruction. The auxiliary pole 10 may comprise a coupler 20 and a shaft 21. The coupler 20 is configured to couple the auxiliary pole 10 to an upright 25 of the IV pole 24. The coupler 20 may comprise a recess 26 configured to be removably disposed over a free end of the upright 25. In some embodiments, the coupler 20 may be configured to couple the auxiliary pole 10 to the upright 25 adjacent to a base 27 of the IV pole 24 or anywhere along the length of the upright 25. The shaft 21 is coupled to the coupler 20. The shaft 21 may extend downward from the coupler 20 when the auxiliary pole 10 is disposed at the top of the upright 25. Alternatively, the shaft 21 may extend upward from the coupler 20 when the auxiliary pole 10 is disposed adjacent to the base 27 of the IV pole 24. The shaft 21 is not in alignment with an axis of the upright 25 when the auxiliary pole 10 is coupled to the upright 25.

The auxiliary IV pole 10 may be configured to facilitate covering of the IV pole 24. A healthcare worker may cover the upright 25 of the IV pole 24 using any of the IV pole isolation devices previously described. The upright 25 may be free from obstructions, such as equipment clamps coupled to the upright 25. The isolation device may fully cover the upright 25. The auxiliary pole 10 is coupled to the upright 25. Medical equipment is coupled to the auxiliary pole 10.

Without further elaboration, it is believed that one skilled in the art may use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art, and having the benefit of this disclosure, that changes may be made to the details of the above-described embodiments without departing from the underlying principles of the disclosure herein.

1. An isolation device for an elongate medical appliance comprising:
a cover disposed over the elongate medical appliance, wherein the cover is configured to isolate the elongate medical appliance from contamination.

2. The isolation device of claim 1, wherein the cover comprises any one of an antimicrobial agent, an antiviral agent, an antifungal agent, and any combination thereof.

3. The isolation device of claim 2, wherein any one of the antimicrobial agent, the antiviral agent, the antifungal agent, and any combination thereof is disposed within a polymeric layer coupled to an inside and/or an outside surface of the cover.

4. The isolation device of claim 1, wherein the cover comprises:
a support apparatus configured to couple with an end of an IV pole; and
a sleeve configured to be deployed from the support apparatus over the IV pole.

5. The isolation device of claim 4, wherein the support apparatus comprises a cavity configured to retain the sleeve prior to deployment over the IV pole.

6. The isolation device of claim 4, wherein the sleeve comprises transverse perforations configured to separate portions of the sleeve.

7. The isolation device of claim 1, wherein the cover comprises:
a ring-shape dispenser configured to be coupled to and at least partially surround an IV pole; and
a sleeve configured to be deployed from the ring-shape dispenser over the elongate medical device.

8. The isolation device of claim 7, wherein the ring-shape dispenser comprises a cavity configured to retain the sleeve prior to deployment over the elongate medical appliance.

9. The isolation device of claim 7, wherein the sleeve is configured to extend from the dispenser and at least partially cover the dispenser.

10. The isolation device of claim 7, wherein the sleeve comprises transverse perforations configured to separate portions of the sleeve.

11. The isolation device of claim 7, wherein the dispenser comprises a notch configured to fit over the elongate medical appliance.

12. The isolation device of claim 7, wherein the sleeve comprises at least one tie partially detachable from the sleeve.

13. The isolation device of claim 12, wherein the at least one tie comprises an adhesive film.

14. The isolation device of claim 12, wherein the at least one tie comprises teeth configured to engage a slit in the sleeve.

15. A method of isolating an elongate medical appliance comprising:
obtaining an isolation cover, and
deploying the isolation cover over the elongate medical appliance.

16. The method claim 15, further comprising:
obtaining the isolation cover comprising a support apparatus and a sleeve;
disposing the support apparatus over an end of the elongate medical appliance; and
extending the sleeve from the support apparatus over the elongate medical appliance.

17. The method of claim 16, further comprising removing a portion of the sleeve at a transverse perforation

18. The method claim 15, further comprising:
obtaining the isolation cover comprising a ring-shape dispenser and a sleeve;
disposing the dispenser at first end of the elongate medical appliance;
extending the sleeve from the dispenser to a second end of the elongate medical appliance such that the sleeve at least partially covers the dispenser and the elongate medical appliance.

19. The method of claim 18, further comprising removing a portion of the sleeve at a transverse perforation.

20. The method of claim 18, further comprising coupling the sleeve to the elongate medical appliance utilizing at least one tie.
